(19) United States
(12) Patent Application Publication
(10) Pub. No.: US 2023/0313280 A1
(43) Pub. Date: Oct. 5, 2023

(54) INTEGRATED NUCLEIC ACID LOOP-MEDIATED ISOTHERMAL AMPLIFICATION AND MOBILE DEVICE SYSTEM

(71) Applicant: National Taiwan University, Taipei (TW)

(72) Inventors: Chien-Fu CHEN, Taipei (TW); Shou-Cheng WU, Taipei (TW); Shi-Jia CHEN, Taipei (TW); Yuh-Shiuan CHIEN, Taipei (TW); Wang-Huei SHENG, Taipei (TW)

(21) Appl. No.: 18/046,575

(22) Filed: Oct. 14, 2022

(30) Foreign Application Priority Data
Mar. 31, 2022 (TW) 111112573

(51) Int. Cl. C12Q 1/6844; B01L 7/00

(52) U.S. Cl. CPC C12Q 1/6844; B01L 7/52; C12Q 2527/101; C12Q 2563/107

(57) ABSTRACT

An integrated nucleic acid loop-mediated isothermal amplification and mobile device system is provided and includes a main body and a power supply, where the 5 main body at least has a delivery unit, a heating unit and a control unit, the control unit is electrically connected to the delivery unit and the heating unit, and the power supply is electrically connected to the main body. A method for operating the integrated nucleic acid loop-mediated isothermal amplification and mobile device system is also provided.

### BACKGROUND

### 1. Technical Field

The present disclosure relates to a system for carrying out a loop-mediated isothermal amplification (LAMP) of nucleic acid, and more particularly, to an integrated nucleic acid loop-mediated isothermal amplification test strip and mobile device system.

### 2. Description of Related Art

At present, the rapid diagnosis of infectious pathogens is mostly carried out by using Enzyme-Linked Immunosorbent Assay (ELISA) or Lateral flow immunoassay (LFA). Among them, LFA is widely used due to its low cost, ease of use, and rapid interpretation of results. However, compared with ELISA, the sensitivity and specificity of LFA are significantly lower, which makes the accuracy of LFA in the rapid diagnosis of infectious pathogens questioned. ELISA uses fluorescent antibodies to bind to proteins on the surface of bacteria or viruses in a direct or indirect manner to achieve the purpose of detection. Although ELISA has higher sensitivity than LFA, its sensitivity is still lower than the sensitivity of polymerase chain reaction (PCR), and it cannot detect the presence of extremely small amounts of bacteria or viruses.

The sensitivity of PCR and Loop-Mediated Isothermal Amplification (LAMP) technology is quite high, but the operation steps are all more complicated, and special technical personnel are required to operate in the laboratory, the pathogen cannot be detected immediately, especially PCR requires expensive temperature cycling equipment to complete the detection. Combining the shortcomings of the above detection methods, LAMP technology is the most popular detection method in recent years, its advantages are not only high specificity and high sensitivity, but it also can be carried out at a constant temperature of 60° C. to 70° C. without precise temperature control, so equipment requirements for LAMP technology are relatively low.

LAMP technology is a new nucleic acid molecule detection technology published in 2000 by Japanese scholar Notomi et al. The main difference between LAMP technology and PCR technology is that the former can carry out the reaction at a constant temperature of 60° C. to 70° C. The LAMP reaction requires three sets of primers, namely outer primers, inner primers and loop primers. The primers are designed for the specific region of the nucleic acid sequence of the sample to be detected, so that the LAMP reaction has a high degree of specificity. In addition to the primers in the LAMP reaction, Bst DNA polymerase should be added, which is an enzyme purified and produced from a bacterium (Bacillus stearothermophilus). It can polymerize with the above-mentioned primers at an optimum temperature (60° C.-65° C.) to amplify DNA fragments with self-complementary ability, form a dumbbell-shaped structure with a stem-loop, whereby the continuous and repeated reaction produces a large number of DNA fragments. During the reaction, there will be the release of pyrophosphate, which will form a white magnesium pyrophosphate precipitate with the magnesium ions in the reaction solution. With the increase of reaction time, a large number of white precipitates can be observed with the naked eye, which means that the LAMP reaction detects the target. In addition, nucleic acid stains ethidium bromide or SYBR Green I can also be added, and then irradiated with ultraviolet light (UV) to observe the reaction results. The effect is better than directly observing the white precipitate with the naked eye.

At present, many LAMP small portable detectors have been developed, but the following problems still need to be solved: (1) the nucleic acid amplification reaction reagents cannot be integrated and prepared in advance. This factor will lengthen the overall detection time. The reagents configured for each detection will also cause errors in the comparison between the results. At the same time, the deployment of the reagents requires specialized technical personnel; (2) the interpretation of the results needs to be verified by indirect methods, such as further analysis of the amplification products with LFA, or measurement of the hydrogen ions released during the amplification reaction as the basis for interpretation; and (3) the detection time is too long, and most of it takes 40 to 60 minutes.

In view of the above-mentioned needs, the present disclosure provides an integrated nucleic acid loop-mediated isothermal amplification and mobile device system that simplifies the element system, does not need to use additional pump-driven reaction reagents, has low element assembly costs, and can be driven by batteries such as mobile power banks.

### SUMMARY

The present disclosure provides an integrated nucleic acid loop-mediated isothermal amplification and mobile device system, comprising: a main body comprising: a delivery unit comprising a pressure roller and a test strip rotating roller arranged opposite to each other to form a gap therebetween, wherein the delivery unit delivers a test strip with a plurality of compartments, such that the plurality of compartments of the test strip are squeezed and destroyed by the pressure roller and the test strip rotating roller when the test strip passes through the gap; a heating unit comprising a ceramic heating chip and a temperature sensor to provide a temperature suitable for performing a loop-mediated isothermal amplification reaction; and a control unit electrically connected to the delivery unit and the heating unit to control operations of the delivery unit and the heating unit; and a power supply electrically connected to the main body to drive the operations of the delivery unit, the heating unit and the control unit.

In at least one embodiment, the plurality of compartments in the test strip include a first compartment located at one end of two sides of the test strip, wherein the first compartment is used for injecting a sample to be detected. In some embodiments, the sample to be detected is selected from one of DNA, RNA, blood, urine, body fluids, feces, secretions and tissues. In at least one embodiment, the sample to be detected is DNA or RNA.

In at least one embodiment, the plurality of compartments in the test strip include a second compartment, a third compartment and a fourth compartment for placing a reagent, a primer and a polymerase, respectively.

In at least one embodiment, components of the reagent comprise dNTP, buffer solution and magnesium sulfate.

In at least one embodiment, the primer is designed according to a nucleic acid sequence of the sample to be detected.

In some embodiments, the polymerase is selected from a group consisting of Bst DNA polymerase, TwistAmp nucleic acid amplification special polymerase, Taq DNA polymerase, AmpliTaq DNA polymerase, DNA polymerases I to V and other DNA or RNA polymerases used in nucleic acid amplification reactions. In at least one embodiment, the polymerase is Bst DNA polymerase.

In at least one embodiment, the plurality of compartments of the test strip further include a fifth compartment, wherein the fifth compartment is used for placing a marker for marking a product obtained by the loop-mediated isothermal amplification reaction.

In some embodiments, the marker is selected from a group consisting of ethidium bromide, SYBR Gold, SYBR Safe, Eva Green and SYBR Green I. In at least one embodiment, the marker is ethidium bromide or SYBR Green I.

In at least one embodiment, the delivery unit further includes a stepping motor, a lever structure block, a compression spring and an anti-slip silicone cover, wherein an output end of the stepping motor is connected to the test strip rotating roller to drive the test strip rotating roller to rotate, the lever structure block is connected to the pressure roller and the compression spring, and the anti-slip silicone cover is disposed outside the test strip rotating roller to adjust a travel path of the test strip in the delivery unit.

In some embodiments, the temperature is between 4° C. and 95° C., such as 4, 5, 6, 7, 8, 9, 10, 15, 20, 25, 30, 35, 40, 45, 50, 55, 60, 65, 70, 75, 80, 85, 90, or 95° C.; in other embodiments, the temperature is between 60° C. and 70° C., and may also be between 66° C. and 69° C., for example, the temperature is 68° C.

In some embodiments, the loop-mediated isothermal amplification reaction time is between 30 minutes and 3 hours, for example, 30 minutes, 1 hour, 2 hours, or 3 hours. In some embodiments, the loop-mediated isothermal amplification reaction time is between 30 minutes and 1 hour.

In at least one embodiment, the power supply can be a generator or a battery. In an embodiment, the power supply is a battery such as a mobile power bank.

In at least one embodiment, the system further comprises a camera unit and an ultraviolet light source. In an embodiment in which the system does not have a camera unit and an ultraviolet light source, after the test strip is withdrawn from the system, the test strip is irradiated with the ultraviolet light source, and the camera unit is used to photograph the fluorescence generated by the reaction between the marker and the product to interpret the results.

The present disclosure further provides a method for operating the integrated nucleic acid loop-mediated isothermal amplification and mobile device system according to the present disclosure, comprising: taking out the test strip that has been pre-prepared and stored in a temperature range between −80° C. and 40° C. to thaw a reagent, wherein the test strip has a first compartment, a second compartment, a third compartment and a fourth compartment, and wherein the second compartment, the third compartment and the fourth compartment are placed with the reagent, a primer and a polymerase, respectively; injecting a sample to be detected into the first compartment in the test strip after thawing; placing the test strip injected with the sample to be detected in the delivery unit, wherein the pressure roller and the test strip rotating roller are driven to squeeze the test strip via the control unit to destroy the first compartment to the fourth compartment and allow the reagent, the polymerase, the primer and the sample to be detected to be mixed; using the heating unit to heat the test strip after mixing the reagent, the polymerase, the primer and the sample to be detected for enabling the reagent, the polymerase, the primer and the sample to be detected to undergo the loop-mediated isothermal amplification reaction; driving the pressure roller and the test strip rotating roller via the control unit to gather a product obtained from the loop-mediated isothermal amplification reaction in the test strip after completing the loop-mediated isothermal amplification reaction; and withdrawing the test strip from the system.

In some embodiments, a temperature for performing the loop-mediated isothermal amplification reaction is between 4° C. and 95° C., such as 4, 5, 6, 7, 8, 9, 10, 15, 20, 25, 30, 35, 40, 45, 50, 55, 60, 65, 70, 75, 80, 85, 90, or 95° C.; in other embodiments, the temperature is between 60° C. and 70° C., and may also be between 66° C. and 69° C., for example, the temperature is 68° C.

In some embodiments, a time for performing the loop-mediated isothermal amplification reaction is between 30 minutes and 3 hours, for example, 30 minutes, 1 hour, 2 hours, or 3 hours. In some embodiments, the loop-mediated isothermal amplification reaction time is between 30 minutes and 1 hour.

In at least one embodiment, the test strip further includes a fifth compartment for placing a marker for marking the product obtained by the loop-mediated isothermal amplification reaction, wherein the method further comprises driving the pressure roller and the test strip rotating roller via the control unit to destroy the fifth compartment containing the marker in the test strip after performing the loop-mediated isothermal amplification reaction, such that the marker and the product are mixed and gathered, and then the test strip is withdrawn from the system.

In at least one embodiment, the method further comprises placing the test strip in which the marker and the product are mixed under an ultraviolet light source, and photographing a fluorescent light generated by a reaction of the marker and the product with a camera unit to interpret results.

The integrated nucleic acid amplification reaction test strip in the integrated nucleic acid loop-mediated isothermal amplification and mobile device system according to the present disclosure contains pre-prepared all-in-one reagents, which greatly shortens the pre-detection processing procedure, and also solves the problem of requiring specialized technical personnel to prepare.

Moreover, the integrated nucleic acid loop-mediated isothermal amplification and mobile device system according to the present disclosure is light in size, easy to carry, easy to operate, and has the characteristics of high detection sensitivity and direct interpretation of analytical values.

Further, the integrated nucleic acid loop-mediated isothermal amplification and mobile device system according to the present disclosure is used for the loop-mediated isothermal amplification of the sample to be detected, which can be completed in 30 minutes, and the primer can be flexibly changed to detect different pathogens.

### BRIEF DESCRIPTION OF THE DRAWINGS

FIG. 1 is a schematic block diagram of an integrated nucleic acid loop-mediated isothermal amplification and mobile device system according to one embodiment of the present disclosure.

FIG. 2 is a schematic side cross-sectional view of a test strip according to one embodiment of the present disclosure.

FIG. 3 is a schematic cross-sectional view of a delivery unit and a heating unit according to one embodiment of the present disclosure.

FIG. 4 is a schematic diagram of the operation flow of the integrated nucleic acid loop-mediated isothermal amplification and mobile device system according to one embodiment of the present disclosure.

FIG. 5A and FIG. 5B show the comparison of the detection performance of the integrated nucleic acid loop-mediated isothermal amplification and mobile device system of the present disclosure and the current commercial PCR machine. FIG. 5A is the fluorescence intensity and electrophoresis of the products obtained after the loop-mediated isothermal amplification of template sequences with different concentrations under the same reaction conditions, and FIG. 5B is a bar graph obtained by quantifying the fluorescence intensity of the products obtained after the loop-mediated isothermal amplification reaction. It can be confirmed from the results in FIG. 5A and FIG. 5B that there is no difference in the performance of the nucleic acid amplification reaction between the system of the present disclosure and the commercially available PCR machine.

FIG. 6A and FIG. 6B show the detection results of the shelf life of the test strip of the present disclosure under the conditions of different storage temperatures and different storage times. FIG. 6A shows the fluorescence intensity and electrophoresis of the products obtained after the test strip of the present disclosure is stored at −20° C. and 4° C. for one to five weeks, and the template sequence of the same concentration is subjected to the loop-mediated isothermal amplification under the same conditions, and FIG. 6B is a bar graph obtained by quantifying the fluorescence intensity of the products obtained after the loop-mediated isothermal amplification reaction. It can be found from the results in FIG. 6A and FIG. 6B that the test strip of the present disclosure can still effectively amplify the template sequence when stored at −20° C. for five weeks, while the test strip of the present disclosure stored at a temperature of 4° C. cannot effectively maintain its efficiency of amplifying the template sequence after three weeks.

### DETAILED DESCRIPTIONS

The following describes the implementation of the present disclosure with examples. Those familiar with the art can easily understand the other advantages and effects of the present disclosure from the content disclosed in this specification. However, the embodiments set forth herein are not intended to limit the present disclosure, and the present disclosure can also be implemented or applied by other different embodiments, and the details set forth herein can also be based on different viewpoints and applications. Various changes or modifications are given without going beyond the spirit of the present disclosure.

The structures, ratios, sizes, and the like in the accompanying figures are used to illustrate the content disclosed in the present disclosure for one skilled in the art to read and understand, rather than to limit the conditions for practicing the present disclosure. Any modification of the structure, alteration of the ratio relationship, or adjustment of the size without affecting the possible effects and achievable proposes should still fall in the range compressed by the technical content disclosed in the present disclosure.

When “comprising,” “including” or “having” an element described herein, unless otherwise specified, other requirements such as elements, components, structures, regions, parts, devices, systems, steps, or connection relationships may be further included, rather than excluding these other requirements.

Terms such as “upper,” “lower,” “top,” “bottom,” “side,” “front,” “rear” and the like used herein are merely used for clear explanation rather than limiting practical range by the present disclosure, and thus, the adjustment, exchange, or alteration of relative relationship thereof without altering the technical content should be considered in the practical scope of the present disclosure.

The sequential terms such as “first” or “second” described herein are for convenience in describing or distinguishing elements, components, structures, regions, parts, devices, systems and other requirements. It is not intended to limit the scope of implementation of the present disclosure, nor to limit the spatial order of these requirements. In addition, the singular forms “a” and “the” described herein also include the plural unless the context clearly dictates otherwise, and “or” is used interchangeably with “and/or” as used herein.

The numerical ranges stated herein are inclusive and combinable, and any numerical value falling within the numerical ranges stated herein should be included within the scope of the present disclosure, and may also be used as the maximum value or minimum value to derive a sub-range. For example, the numerical range of “60° C. to 70° C.” should be understood to include 60° C., 60.5° C., 61° C., 61.5° C., 62° C., 62.5° C., 63° C., 63.5° C., 64° C., 64.5° C., 65° C., 65.5° C., 66° C., 66.5° C., 67° C., 67.5° C., 68° C., 68.5, 69° C., 69.5° C., and 67° C., and also include any sub-range between the minimum value of 60° C. and the maximum value of 70° C., e.g.: 60° C. to 69° C., 61° C. to 70° C., etc.

The term “gather” described herein refers to the technical means for ensuring the diffusion of the liquid flow of the test strip reaction reagent. Since the structure of the test strip of the present disclosure is a flat space, the reaction reagent will be diffused and distributed due to space squeeze. Therefore, after the test strip reaction is completed and exits the device system, it is necessary to have its “gather” step. The technical means of the “gather” step described herein is to use the delivery unit of the present disclosure or the user's fingers to squeeze and push the reaction reagent to the front end of the test strip, and then use the QR barcode sample identification sticker to fix the gather of the reagent.

The first embodiment of the present disclosure is an integrated nucleic acid loop-mediated isothermal amplification and mobile device system, comprising a main body and a power supply, wherein the main body at least has a delivery unit, a heating unit and a control unit, wherein the delivery unit, the heating unit and the control unit are arranged inside the main body, the control unit is electrically connected to the delivery unit and the heating unit, and the power supply is electrically connected to the main body.

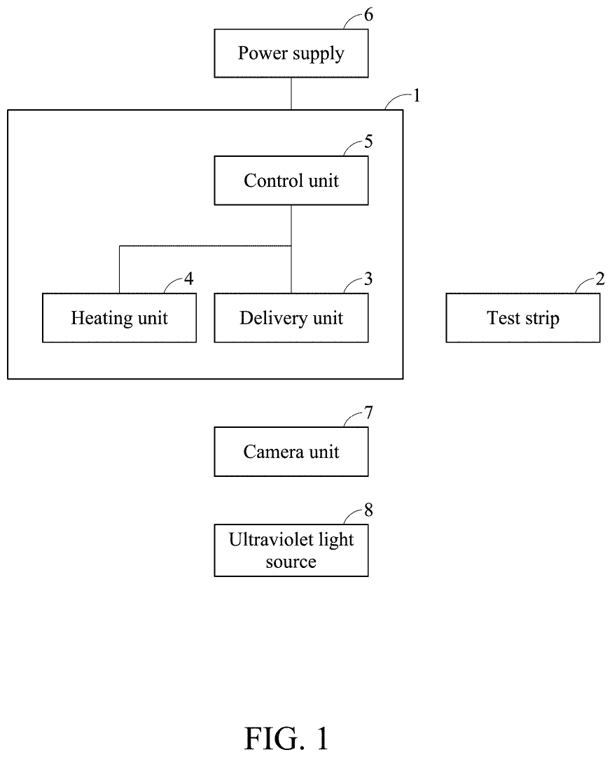

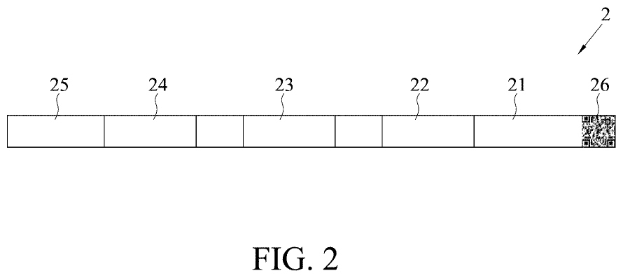

In at least one embodiment, the integrated nucleic acid loop-mediated isothermal amplification and mobile device system of the present disclosure is shown in FIG. 1, wherein a main body 1 includes a delivery unit 3, a heating unit 4 and a control unit 5, wherein the delivery unit 3 is used to deliver a test strip 2 having a plurality of compartments and containing the reagent, the polymerase, the primer and the sample to be detected for the loop-mediated isothermal amplification reaction, and the delivery unit 3 is provided with a plurality of rollers for squeezing and destroying the plurality of compartments of the test strip 2, such as a first compartment 21, a second compartment 22, a third compartment 23 and a fourth compartment shown in FIG. 2, so as to mix the reagent, the polymerase, the primer and the sample to be detected; the heating unit 4 is used to provide a temperature suitable for the reagent, the polymerase, the primer and the sample to be detected to perform the loop-mediated isothermal amplification reaction; the control unit 5 is electrically connected to the delivery unit 3 and the heating unit 4 to control operations of the delivery unit 3 and the heating unit 4;

and the power supply 6 is used to drive the operations of the delivery unit 3, the heating unit 4 and the control unit 5, and the power supply 6 is electrically connected to the main body 1.

In one embodiment, the material of the test strip 2 is polypropylene (PP), which is a semi-crystalline thermoplastic. The overall specifications of test strip 2 and the space design for the placement of the contents are shown in FIG. 2. The thickness of the selected material is 0.1 mm, and the test strips with the size of 5 mm (width) and 130 mm (length) are made by a hot-pressing sealing machine. The structure after preparation is in a flat space state, and the enzymes, primers and buffers injected later must be sealed with heat to keep them spaced apart from each other to avoid mixing of the liquid streams. The front and rear ends of the test strips are in a sealed state. When detecting, a gap is cut with scissors and the sample is injected, and then the gap is sealed. In some embodiments, the gap can be sealed using, for example, a QR barcode sample identification sticker.

As shown in FIG. 2, the test strip 2 includes a plurality of compartments, such as the first compartment 21, the second compartment 22, the third compartment 23 and the fourth compartment 24. The first compartment 21 is located at one end of the two sides of the test strip 2. The first compartment 21 is used for injecting the sample to be detected and sealing the test strip with a QR barcode sample identification sticker 26. In at least some embodiments, the sample to be detected is selected from one of DNA, RNA, blood, urine, body fluids, feces, secretions and tissues. In at least one embodiment, the sample to be detected is DNA. In at least another embodiment, the sample to be detected is RNA.

The second compartment 22, the third compartment 23 and the fourth compartment 24 in the test strip 2 are used to pre-insert the reagent, the primer and the polymerase, respectively, wherein the components of the reagent include dNTP, buffer solution and magnesium sulfate, the primer is designed for the nucleic acid sequence of the sample to be detected, and the polymerase is selected from the group consisting of Bst DNA polymerase, TwistAmp nucleic acid amplification special polymerase, Taq DNA polymerase, AmpliTaq DNA polymerase, DNA polymerases I to V and other DNA or RNA polymerases used in nucleic acid amplification reactions. In an embodiment, the polymerase is Bst DNA polymerase.

In one embodiment, the test strip 2 also includes a fifth compartment 25 for pre-inserting a marker for marking the product obtained by the loop-mediated isothermal amplification reaction, wherein the marker is selected from the group consisting of ethidium bromide, SYBR Gold, SYBR Safe, Eva Green and SYBR Green I. In an embodiment, the marker is ethidium bromide or SYBR Green I.

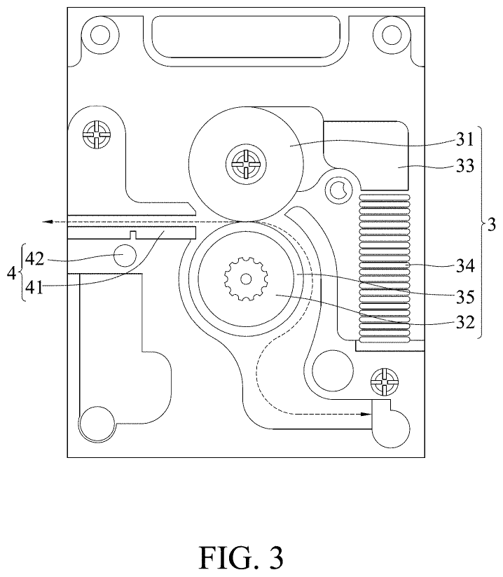

In one embodiment, the delivery unit 3 and the heating unit 4 are integrated in the main body 1. As shown in FIG. 3, the delivery unit 3 mainly includes elements such as a pressure roller 31, a test strip rotating roller 32, a stepping motor (located in the center of the main body 1, not shown in the figure), a lever structure block 33, a compression spring 34 and an anti-slip silicone cover 35, etc., wherein the output end of the stepping motor is connected to the test strip rotating roller 32 as a power source for driving the test strip rotating roller 32 to rotate. The lever structure block 33 is connected to the pressure roller 31 and the compression spring 34. The anti-slip silicone cover 35 is disposed outside the test strip rotating roller 32 to adjust the travel path (the dotted line in FIG. 3) of the test strip 2 in the delivery unit 3. In addition, the heating unit 4 mainly includes elements such as a ceramic heating chip 41 and a temperature sensor 42, which are used to provide a temperature suitable for performing a loop-mediated isothermal amplification reaction.

In one embodiment, the control unit 5 is composed of a control board and a plurality of control buttons, so as to perform various adjustments such as the motor rotation angle of the delivery unit 3, the heating time and the heating temperature of the heating unit 4. In other embodiments, the control board is an Arduino UNO control board.

In one embodiment, the user puts the test strip from the entrance on the left side of the main body 1, as shown in FIG. 3, when the test strip is surely placed in the gap between the pressure roller 31 and the test strip rotating roller 32, press the control button of the control unit 5, so that the stepping motor located in the center of the main body 1 rotates clockwise to roll the test strip into the main body 1. The pressure roller 31 transmits the elastic force of the compression spring 34 via the lever structure block 33 to provide a fixed positive force to help the test strip 2 to be squeezed. During the squeezing process, the hot-pressed seals of the partition walls inside the test strip 2 will be ruptured, allowing the liquid to flow out and achieve the purpose of adequate mixing. After the main body 1 rolls in the entire test strip 2, the reagents inside the test strip 2 will be mixed in the area of the ceramic heating chip 41, and at this time, the ceramic heating chip 41 is automatically turned on for a constant temperature heating. After the heating is completed, the stepping motor will automatically reverse and withdraw the test strip 2 so that the user can take it out.

In some embodiments, the temperature provided by the heating unit 4 is between 4° C. and 95° C. In other embodiments, the temperature provided by the heating unit 4 is between 60° C. and 70° C. In other embodiments, the temperature may be 60° C., 60.5° C., 61° C., 61.5° C., 62° C., 62.5° C., 63° C., 63.5° C., 64° C., 64.5° C., 65° C., 65.5° C., 66° C., 66.5° C., 67° C., 67.5° C., 68° C., 68.5, 69° C., 69.5° C. and 67° C., etc. In at least one embodiment, the temperature is between 66° C. and 69° C. In at least one embodiment, the temperature is 68° C.

In other embodiments, the loop-mediated isothermal amplification reaction time of the present disclosure is between 30 minutes and 3 hours. In other embodiments, the loop-mediated isothermal amplification reaction time of the present disclosure ranges from 30 minutes to 1 hour. In at least one embodiment, the loop-mediated isothermal amplification reaction time of the present disclosure is about 30 minutes.

In one embodiment, the power supply 6 is a mobile power bank. In other embodiments, the power supply 6 can be a generator.

In one embodiment, the integrated nucleic acid loop-mediated isothermal amplification and mobile device system of the present disclosure includes a camera unit 7 and an ultraviolet light source 8.

In one embodiment, since the test strip 2 contains the SYBR Green I oligonucleotide color marker, the color marker will be embedded in the double-stranded helix DNA when the nucleic acid substance undergoes an isothermal amplification reaction. At this time, when irradiated with a light source with a blue light wavelength between 400 nm and 500 nm, green fluorescence can be easily observed with the naked eye.

In one embodiment, the irradiation of the ultraviolet light source 8 and the action of the camera unit 7 and the main body 1 are not involved in the spatial relationship of the operation, wherein the ultraviolet light source 8 only needs an LED lamp with a blue light wavelength between 400 nm and 500 nm to excite the color marker to generate fluorescence. At the same time, the camera unit 7 can take pictures and capture pictures with a general mobile phone camera, and then the results of nucleic acid amplification can be further analyzed by adjusting the picture comparison.

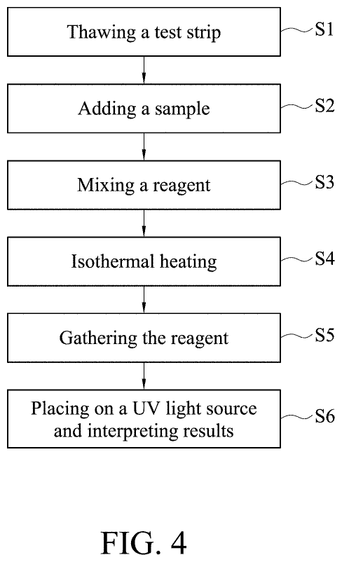

The second embodiment of the present disclosure provides a method for operating the integrated nucleic acid loop-mediated isothermal amplification and mobile device system as described in the present disclosure. In at least one embodiment, the method includes the steps shown in FIG. 4 and is described in the following.

Step 1: taking out the test strip 2 that is pre-prepared and stored at −20° C., and when the reagent is thawed, the test strip 2 has the first compartment 21, the second compartment 22, the third compartment 23 and the fourth compartment 24, wherein the reagent, the primer and the polymerase are respectively placed in the second compartment 22, the third compartment 23 and the fourth compartment 24.

Step 2: injecting the sample to be detected into the first compartment 31 in the test strip 2 after thawing.

Step 3: placing the test strip 2 containing the sample to be detected in the delivery unit 3, wherein, through the control unit 5, the pressure roller and the test strip rotating roller are driven to squeeze the test strip injected with the sample to be detected, so as to destroy the first compartment 21 to the fourth compartment 24 and allow the reagent, the polymerase, the primer and the sample to be detected in each compartment to be mixed.

Step 4: using the heating unit 4 to heat the test strip 2 after mixing the reagent, the polymerase, the primer and the sample to be detected, so that the reagent, the polymerase, the primer and the sample to be detected are underwent a loop-mediated isothermal amplification reaction.

Step 5: after completing the loop-mediated isothermal amplification reaction, driving the pressure roller and the test strip rotating roller through the control unit 5 to gather the reagent, the polymerase, the primer, the sample to be detected and the product obtained from the loop-mediated isothermal amplification reaction in the test strip 2, and then withdrawing the test strip 2 from the system.

In one embodiment, the temperature at which the loop-mediated isothermal amplification reaction is carried out is 68° C. In some embodiments, the temperature is between 66° C. and 69° C.

In one embodiment, the time for carrying out the loop-mediated isothermal amplification reaction is about 30 minutes.

In one embodiment, the test strip 2 further comprises the fifth compartment 25, which is used for inserting a marker for marking the product obtained by the loop-mediated isothermal amplification reaction, and the method further comprises, after performing the loop-mediated isothermal amplification reaction, driving the pressure roller and the test strip rotating roller through the control unit 5 to destroy the third compartment 23 containing the marker in the test strip, so that the marker and the product are mixed and gathered, and then the test strip 2 is withdrawn from the system.

In one embodiment, the method further comprises placing the test strip in which the marker and the product are mixed under the ultraviolet light source, and photographing the fluorescent light generated by the reaction of the marker and the product with a camera unit, to interpret the results.

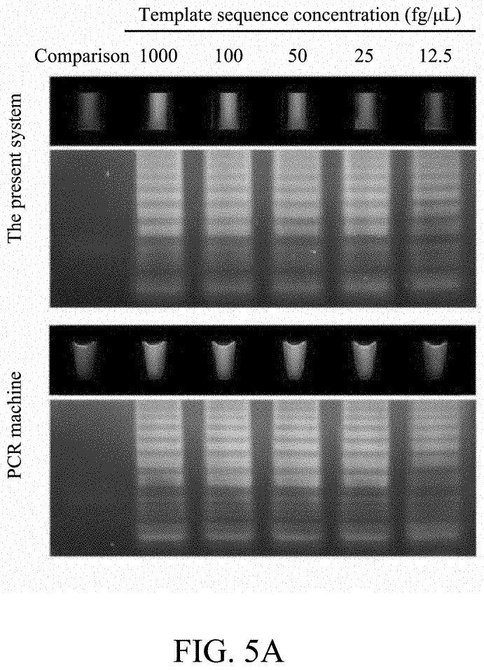

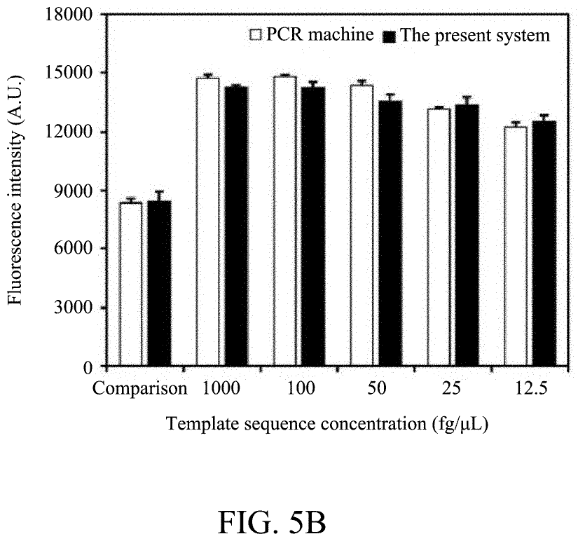

In another embodiment of the present disclosure, the detection performance of the integrated nucleic acid loop-mediated isothermal amplification and mobile device system of the present disclosure and the current PCR machine are compared. FIG. 5A is the fluorescence intensity and electrophoresis of the products obtained after the loop-mediated isothermal amplification of template sequences with different concentrations under the same reaction conditions, and FIG. 5B is a bar graph obtained by quantifying the fluorescence intensity of the products obtained after the loop-mediated isothermal amplification reaction. As shown in FIG. 5A and FIG. 5B, both the system of the present disclosure and the current PCR machine can successfully amplify samples of different concentrations, the product fluorescence detection comparison also confirms that there is no significant difference between the two, thereby indicating that the nucleic acid amplification performance of the device of the present disclosure is no different from that of the current PCR machine.

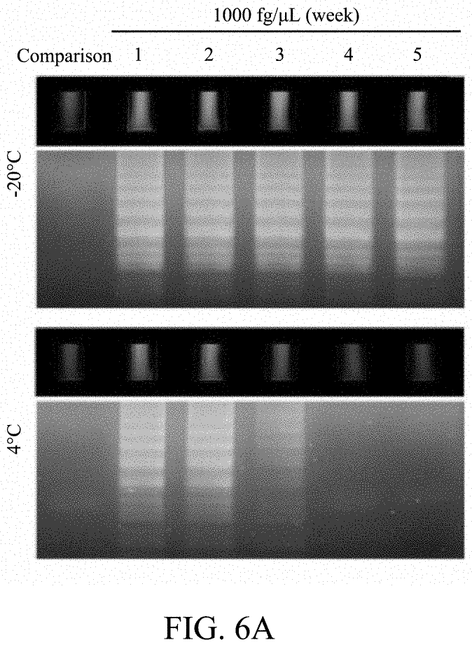

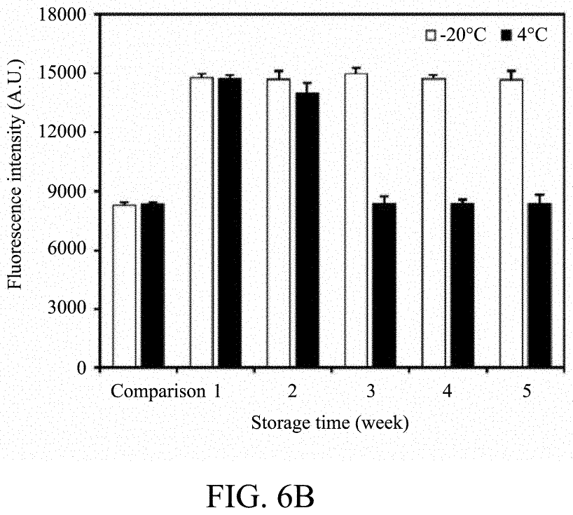

In yet another embodiment of the present disclosure, the test is carried out for the shelf life of the test strip of the present disclosure. FIG. 6A shows the fluorescence intensity and electrophoresis of the products obtained after the test strip of the present disclosure is stored at −20° C. and 4° C. for 1 to 5 weeks, and the template sequence of the same concentration is subjected to the loop-mediated isothermal amplification reaction under the same conditions, and FIG. 6B is a bar graph obtained by quantifying the fluorescence intensity of the products obtained after the loop-mediated isothermal amplification reaction. It can be found from the results in FIG. 6A and FIG. 6B that the test strip of the present disclosure can still effectively amplify the template sequence when stored at −20° C. for 5 weeks, while the test strip of the present disclosure stored at a temperature of 4° C. cannot effectively maintain its efficiency of amplifying the template sequence after 3 weeks. This means that the test strip of the present disclosure can be pre-prepared and stored at −20° C., and there is no need to prepare these reagents before the loop-mediated isothermal amplification reaction by a professional technician.

The test strip of the present disclosure is a reagent storage platform, and the type of the reagent can be replaced by changing the nucleic acid amplification reaction method. The storage conditions of the nucleic acid amplification reaction reagents used at that time include normal temperature, refrigeration and freezing. As mentioned above, the current reagents for nucleic acid amplification reactions also include liquid and solid types with various storage temperatures. Therefore, the test strip of the present disclosure can be stored at a temperature between −80° C. and 40° C. according to actual needs.

In addition, although the loop-mediated isothermal amplification reaction time is shown as 30 minutes in the above-mentioned embodiment, the overall time will vary due to different nucleic acid amplification methods. Currently, common nucleic acid amplification methods include: nucleic acid sequence bases amplification (NASBA), strand displacement amplification (SDA), loop-mediated isothermal amplification (LAMP), recombinase polymerase amplification (RPA), polymerase chain reaction (PCR) and so on. Therefore, when the test strip of the present disclosure is applied to different nucleic acid amplification methods, the time spent is different. Therefore, the loop-mediated isothermal amplification reaction time of the present disclosure can be about 30 minutes to 3 hours according to actual needs.

The test strip of the present disclosure can be applied to different nucleic acid amplification methods, and different nucleic acid amplification methods have its suitable implementation temperature. Therefore, the implementation temperature of the loop-mediated isothermal amplification reaction of the present disclosure can be between about 4° C. and 95° C. according to actual needs.

Compared with many currently conventional LAMP small detectors, the integrated nucleic acid loop-mediated isothermal amplification and mobile device system provided by the present disclosure has the following advantages: (1) the integrated nucleic acid amplification reaction test strip contains pre-prepared all-in-one reagents, which greatly shortens the pre-detection processing procedure, and also solves the problem of requiring specialized technical personnel to prepare; (2) it is light in size, easy to carry, easy to operate, and has the characteristics of high detection sensitivity and direct interpretation of analytical values; and (3) the loop mediated isothermal amplification is completed in 30 minutes, and the primer can be changed flexibly to detect different pathogens.

The foregoing embodiments are used for the purpose of illustrating the principles and effects rather than limiting the present disclosure. Anyone skilled in the art can modify and alter the above embodiments without departing from the spirit and scope of the present disclosure. Therefore, the range claimed by the present disclosure should be as described by the accompanying claims listed below.

What is claimed is:

1. An integrated nucleic acid loop-mediated isothermal amplification and mobile device system, comprising:
a main body comprising:
a delivery unit comprising a pressure roller and a test strip rotating roller arranged opposite to each other to form a gap therebetween, wherein the delivery unit delivers a test strip with a plurality of compartments, such that the plurality of compartments of the test strip are squeezed and destroyed by the pressure roller and the test strip rotating roller when the test strip passes through the gap;
a heating unit comprising a ceramic heating chip and a temperature sensor to provide a temperature suitable for performing a loop-mediated isothermal amplification reaction; and
a control unit electrically connected to the delivery unit and the heating unit to control operations of the delivery unit and the heating unit; and
a power supply electrically connected to the main body to drive the operations of the delivery unit, the heating unit and the control unit.

2. The system of claim 1, wherein the plurality of compartments in the test strip include a first compartment located at one end of two sides of the test strip, wherein the first compartment is used for injecting a sample to be detected, wherein the sample to be detected is selected from one of DNA, RNA, blood, urine, body fluids, feces, secretions and tissues.

3. The system of claim 2, wherein the sample to be detected is DNA or RNA.

4. The system of claim 2, wherein the plurality of compartments in the test strip include a second compartment, a third compartment and a fourth compartment for placing a reagent, a primer and a polymerase, respectively.

5. The system of claim 4, wherein components of the reagent comprise dNTP, buffer solution and magnesium sulfate.

6. The system of claim 4, wherein the primer is designed according to a nucleic acid sequence of the sample to be detected.

7. The system of claim 4, wherein the polymerase is selected from a group consisting of Bst DNA polymerase, TwistAmp nucleic acid amplification special polymerase, Taq DNA polymerase, AmpliTaq DNA polymerase, DNA polymerases I to V and other DNA or RNA polymerases used in nucleic acid amplification reactions.

8. The system of claim 7, wherein the polymerase is Bst DNA polymerase.

9. The system of claim 4, wherein the plurality of compartments of the test strip further include a fifth compartment, wherein the fifth compartment is used for placing a marker for marking a product obtained by the loop-mediated isothermal amplification reaction.

10. The system of claim 9, wherein the marker is selected from a group consisting of ethidium bromide, SYBR Gold, SYBR Safe, Eva Green and SYBR Green I.

11. The system of claim 10, wherein the marker is ethidium bromide or SYBR Green I.

12. The system of claim 1, wherein the delivery unit further includes a stepping motor, a lever structure block, a compression spring and an anti-slip silicone cover, wherein an output end of the stepping motor is connected to the test strip rotating roller to drive the test strip rotating roller to rotate, the lever structure block is connected to the pressure roller and the compression spring, and the anti-slip silicone cover is disposed outside the test strip rotating roller to adjust a travel path of the test strip in the delivery unit.

13. The system of claim 1, wherein the temperature is between 4° C. and 95° C. 14. The system of claim 1, wherein the power supply is a generator or a battery.

15. The system of claim 1, further comprising a camera unit and an ultraviolet light source.

16. A method for operating the system of claim 1, comprising:
taking out the test strip that has been pre-prepared and stored in a temperature range between −80° C. and 40° C. to thaw a reagent, wherein the test strip has a first compartment, a second compartment, a third compartment and a fourth compartment, and wherein the second compartment, the third compartment and the fourth compartment are placed with the reagent, a primer and a polymerase, respectively;
injecting a sample to be detected into the first compartment in the test strip after thawing;
placing the test strip injected with the sample to be detected in the delivery unit, wherein the pressure roller and the test strip rotating roller are driven to squeeze the test strip via the control unit to destroy the first compartment to the fourth compartment and allow the reagent, the polymerase, the primer and the sample to be detected to be mixed;
using the heating unit to heat the test strip after mixing the reagent, the polymerase, the primer and the sample to be detected for enabling the reagent, the polymerase, the primer and the sample to be detected to undergo the loop-mediated isothermal amplification reaction;
driving the pressure roller and the test strip rotating roller via the control unit to gather a product obtained from the loop-mediated isothermal amplification reaction in the test strip after completing the loop-mediated isothermal amplification reaction; and
withdrawing the test strip from the system.

17. The method of claim 16, wherein a temperature for performing the loop-mediated isothermal amplification reaction is between 4° C. and 95° C.

18. The method of claim 16, wherein a time for performing the loop-mediated isothermal amplification reaction is between 30 minutes and 3 hours.

19. The method of claim 16, wherein the test strip further includes a fifth compartment for placing a marker for marking the product obtained by the loop-mediated isothermal amplification reaction, wherein the method further comprises driving the pressure roller and the test strip rotating roller via the control unit to destroy the fifth compartment containing the marker in the test strip after performing the loop-mediated isothermal amplification reaction, such that the marker and the product are mixed and gathered, and then the test strip is withdrawn from the system.

20. The method of claim 19, further comprising placing the test strip in which the marker and the product are mixed under an ultraviolet light source, and photographing a fluorescent light generated by a reaction of the marker and the product with a camera unit to interpret results.
